(19) United States
(12) Patent Application Publication
(10) Pub. No.: US 2026/0027360 A1
(43) Pub. Date: Jan. 29, 2026

(54) MEDICAL DEVICE FOR USE IN PAIN THERAPY

(71) Applicant: B. Braun Melsungen AG, Melsungen (DE)

(72) Inventors: Jens Wildhagen, Hannover (DE); Urs Eichenberger, Dierikon (CH); David Lorenzana, Oberrieden (CH)

(21) Appl. No.: 19/277,683

(22) Filed: Jul. 23, 2025

(30) Foreign Application Priority Data
Jul. 24, 2024 (DE) 10 2024 120 966.6

(51) Int. Cl. A61N 1/36

(52) U.S. Cl. CPC A61N 1/36017; A61N 1/36021; A61N 1/36034

(57) ABSTRACT

A medical device for use in pain therapy includes an invasive component having at least one electrode configured to be positioned at a nerve and to emit an electrical stimulation signal. A signal generator is connected to the electrode and generates the stimulation signal. The stimulation signal is configured to inhibit stimulation transmission of the nerve. The at least one electrode is configured to emit an electrical monitoring signal. The signal generator is configured to generate the monitoring signal. The monitoring signal is configured to trigger a motor and/or sensory reaction, the form of which depends on a distance between the electrode and the nerve. The medical device can be used to apply neurostimulation in pain therapy.

### CROSS-REFERENCE TO RELATED APPLICATION

This application claims priority under 35 U.S.C. § 119 to German Application No. 10 2024 120 966.6, filed on Jul. 24, 2024, the content of which is incorporated by reference herein in its entirety.

### FIELD

The present disclosure relates to a medical device for use in pain therapy, comprising an invasive component having at least one electrode, which is configured to be positioned at a nerve and to emit an electrical stimulation signal, and a signal generator, which is connected to the electrode and is configured to generate the stimulation signal, wherein the stimulation signal is configured to inhibit stimulation transmission of the nerve.

### BACKGROUND

Such devices are known in the prior art and are used to treat acute or chronic pain in that the transmission of nerve pulses is inhibited by the emission of an electrical stimulation signal in order to alleviate the sensation of pain of the patient. For this purpose, the invasive component of the medical device is introduced into the body, wherein the electrode arranged or formed on the invasive component is positioned close to the nerve. The electrical stimulation signal is generated by means of the signal generator of the medical device and emitted via the electrode.

The position of the electrode in relation to the nerve to be stimulated is crucial for the achievable therapy effect. If the electrode moves closer to the nerve during the stimulation procedure, the stimulation energy emitted at the nerve increases. If the electrode moves away, in this way the stimulation energy acting on the nerve is reduced. Such distance changes and accompanying changes of the stimulation energy can be caused, for example, by movements of the patient.

Methods for regulating the emitted stimulation energy are already known from the prior art. These methods provide tracking and correction of the stimulation energy and are used in the field of neurostimulation in the spinal canal (SCS). For this purpose, for example, the “coupling” of the stimulation signal at the nerve and its response are measured. The stimulation energy of the electrical stimulation signal is adjusted depending thereon.

The response of the nerve can only be measured in the case of direct contact of two electrodes with the nerve. Continuous contact of the electrode or the electrodes with the nerve cannot be assumed in the stimulation of peripheral nerves in the so-called single shot technique.

### SUMMARY

It is an object of the present disclosure to provide a medical device of the type mentioned at the outset, which enables improved pain therapy.

This object is achieved in that the at least one electrode is configured to emit an electrical monitoring signal and that the signal generator is configured to generate the monitoring signal, wherein the monitoring signal is configured to generate a motor and/or sensory reaction, the form of which depends on a distance between the electrode and the nerve. The medical device according to the present disclosure therefore permits the generation and emission of two signals, namely, on the one hand, the electrical stimulation signal and, on the other hand, the electrical monitoring signal. The stimulation signal is used for the actual pain suppression in that it inhibits the stimulation transmission of the affected nerve. The monitoring signal is used for monitoring the position or else monitoring the distance of the electrode in that it is configured to trigger a motor and/or sensory reaction of the patient, wherein its form depends on the distance between the electrode and the nerve. Depending on the form of the motor and/or sensory reaction, the medical personnel carrying out the pain therapy (in the case of a sensory reaction by feedback of the patient themselves) can recognize whether the electrode has to be repositioned to enable optimum pain therapy. In the case of a weakly pronounced motor and/or sensory reaction, a further distal advance of the electrode in the direction of the nerve will regularly be necessary. In the case of a strongly or excessively strongly pronounced motor and/or sensory reaction, a proximal retraction of the electrode to increase the distance will regularly be necessary. The form of the motor and/or sensory reaction can also depend on an intensity of the monitoring signal. In the case of a strongly or excessively strongly pronounced motor and/or sensory reaction, an adaptation, especially reduction, of the intensity of an overall signal consisting of stimulation signal and monitoring signal can be necessary. The electrical stimulation signal for the actual pain suppression has specific properties, especially electrical and/or signalling properties. These properties are known to a person skilled in the art active in the relevant technical field, so that an express specification of the mentioned properties is not necessary at this point. The electrical monitoring signal has different properties, especially different electrical and/or different signalling properties. In one embodiment, the invasive component is a catheter to which the at least one electrode is attached. In a further embodiment, the invasive component is a stimulation cannula to which the at least one electrode is attached. In a further embodiment, the invasive component is a wire and so to speak forms the at least one electrode or vice versa. In one embodiment, the medical device has a single electrode, which is configured both to emit the stimulation signal and to emit the monitoring signal. In a further embodiment, at least two electrodes are present, wherein a first of the two electrodes is configured to emit the stimulation signal and a second of the two electrodes is configured to emit the monitoring signal. Alternatively or additionally, a first electrode of the at least two electrodes can be used as the cathode and a second electrode of the at least two electrodes can be used as the anode. The invasive component including the electrode is configured to be introduced into the body of the patient. In contrast, the signal generator is an extracorporeal component of the medical device which is connected in a wired manner to the at least one electrode of the invasive component. In other words: The signal generator is intended to remain outside the patient body. In one embodiment, the medical device has a detection apparatus which is configured to detect the motor and/or sensory reaction triggered by means of the monitoring signal and especially its form. Such a detection apparatus is moreover configured in a further embodiment to generate a signal depending on the detected form of the motor and/or sensory reaction, which represents the form of the motor and/or sensory reaction. The signal is preferably an acoustic or optical signal and/or a signal which is perceptible by medical personnel in another manner and on the basis of which the medical personnel can decrease or increase the distance of the electrode to the nerve. In a further embodiment, no such detection apparatus is provided. Instead, the form of the motor and/or sensory reaction is detected by the medical personnel themselves, for example in a visual or tactile manner and/or in another manner.

In one embodiment, the electrical stimulation signal is configured such that it triggers no, in particular no practically significant, motor and/or sensory reaction, and the monitoring signal is configured such that it does not generate inhibition of the stimulation transmission. In other words: In this embodiment, there is no or in any case no practically significant intersection with respect to the physiological reaction(s) generated by the relevant signal. In this embodiment, the stimulation signal has at most a minor, preferably no, influence on the motor and/or sensory reaction. This prevents the monitoring signal from being superimposed on the effect of the stimulation signal and impairing it in this way in an undesired manner. Vice versa, the stimulation signal is also prevented from impairing and corrupting the effect of the monitoring signal.

In a further embodiment, the stimulation signal has a frequency which is greater by at least a factor of 0.5×10³, preferably at least 1×10³, more preferably 1.5×10³, than a frequency of the monitoring signal. In this embodiment, the stimulation signal is high frequency in relation to the monitoring signal. Vice versa, the monitoring signal is low frequency in relation to the stimulation signal. Such a frequency selection can prevent undesired overlaps from occurring in the physiological effect (on the one hand, inhibition of the stimulation transmission, on the other hand, triggering of the motor and/or sensory reaction).

In a further embodiment, a/the frequency of the stimulation signal and a/the frequency of the monitoring signal are similar. The frequencies preferably differ by a factor of at most 5, more preferably of at most 2, more preferably of at most 1.1. In this case, the stimulation signal is generated such that it triggers no or in any case no practically significant motor and/or sensory reaction, i.e. lies below a perception threshold.

In a further embodiment, the monitoring signal has a frequency of 0.1 Hz to 10 Hz, preferably of 0.5 Hz to 5 Hz, more preferably of 1 Hz to 2 Hz, and/or the stimulation signal has a frequency of at least 5 kHz, preferably of at least 10 kHz, more preferably of at least 20 kHz. The abovementioned value ranges for the frequencies of the monitoring signal and the stimulation signal have been found to be particularly advantageous. The above value ranges for the frequency of the monitoring signal are advantageous in particular if a motor reaction is to be triggered (motor monitoring signal). If a sensory reaction/perception is to be triggered (sensory monitoring signal), the monitoring signal preferably has a frequency of 3 Hz to 5 Hz.

In a further embodiment, the stimulation signal has a frequency of 0.1 Hz to 200 Hz, preferably of 0.5 Hz to 50 Hz, more preferably of 3 Hz to 5 Hz, in particular wherein the stimulation signal has a signal strength which lies below the perception threshold.

In a further embodiment, the monitoring signal has an amplitude of 0.01 mA to 20 mA, preferably of 0.1 mA to 10 mA, more preferably of 0.5 mA to 3 mA. The abovementioned value ranges for the amplitude of the amperage of the monitoring signal have been found to be particularly advantageous. The above value ranges for the amplitude of the monitoring signal are advantageous in particular if a motor reaction is to be triggered (motor monitoring signal). If a sensory reaction/perception is to be triggered (sensory monitoring signal), the monitoring signal preferably has an amplitude of 0.03 mA to 60 mA, preferably of 0.3 mA to 30 mA, more preferably of 1.5 mA to 9 mA.

In a further embodiment, the signal generator is configured to generate the monitoring signal as a pulsed signal and to generate the stimulation signal as a pulsed signal. In this case, the stimulation signal is generated at an amplitude below the perception threshold, i.e. so that it triggers no or in any case no practically significant motor and/or sensory reaction.

In a further embodiment, the signal generator is configured to generate the monitoring signal as a pulsed signal and to generate the stimulation signal as a continuous signal. The stimulation signal is preferably generated as a broadband and/or high-frequency signal. In this embodiment, time-continuous emission of the stimulation signal is therefore provided, whereas the monitoring signal is emitted in a pulsed and therefore time-discontinuous manner or else at discrete times. Such continuous emission of the stimulation signal, on the one hand, and pulsed emission of the monitoring signal, on the other hand, has been found to be particularly advantageous.

In a further embodiment, the monitoring signal has a pulse width of 0.01 ms to 20 ms, preferably of 0.05 ms to 10 ms, more preferably of 0.1 ms to 1 ms. The abovementioned value ranges for the pulse width of the monitoring signal are accompanied with special advantages. The above value ranges for the pulse width of the monitoring signal are advantageous in particular if a motor reaction is to be triggered (motor monitoring signal). If a sensory reaction/perception is to be triggered (sensory monitoring signal), the monitoring signal preferably has a pulse width of 0.2 ms.

In a further embodiment, the signal generator is configured to generate the monitoring signal in at least one burst having a number of pulses of 1 to 100 pulses, preferably of 1 to 10 pulses, more preferably of 1 to 5 pulses. In this embodiment, intermittent generation and emission of the monitoring signal are therefore provided. The generation and emission take place in the form of at least one burst, i.e. a sequence of single pulses, wherein the sequence has a defined number of single pulses. The pulses preferably each have an identical pulse width, i.e. pulse duration. More preferably, a defined (short) pause duration is provided between each of the pulses. If multiple successive bursts are generated and emitted, a (longer) pause duration provided between the bursts is provided. In this embodiment of the present disclosure, the generation and emission of the monitoring signal in relation to the stimulation signal take place chronologically in a very limited manner. In this way, it is possible to prevent in a still further improved manner that an undesired superposition of the physiological reactions generated by means of the two signals occurs. In addition, it is not possible to preclude under all circumstances that the monitoring signal and the motor and/or sensory reaction triggered thereby induces a certain discomfort in the patient. Such potentially unpleasant side effects of the monitoring signal for the patient can be reduced to a minimum by the chronologically limited/restricted signal emission in the form of the at least one pulse and/or one burst.

In a further embodiment, the signal generator is configured to generate the monitoring signal in at least two successive bursts, wherein a pulse duration between the bursts is at least 10 s, preferably at least 15 s, more preferably at least 20 s. Excessive strain of the patient by the monitoring signal and by the motor and/or sensory reactions triggered by means of the monitoring signal is avoided by such a selection of the pause duration. Moreover, this embodiment of the present disclosure proceeds from the consideration that monitoring of the distance of the electrode does not necessarily have to take place in a time-continuous manner. Monitoring at certain time intervals will regularly already be sufficient. This embodiment permits monitoring at such time intervals, wherein the time intervals for the monitoring of the distance are defined by the pause duration between the bursts.

In a further embodiment, the signal generator is configured to generate the monitoring signal and the stimulation signal alternately. The electrode is configured for alternating or simultaneous emission of the two signals in this case.

In a further embodiment, the signal generator is configured to generate the monitoring signal and the stimulation signal simultaneously. In this embodiment, the electrode is accordingly also configured for simultaneous emission of the two signals.

The present disclosure also relates to a method for distance monitoring of an electrode during pain therapy by means of electrical neurostimulation. The method according to the present disclosure comprises the following steps: generating an electrical monitoring signal, wherein the monitoring signal is generated by means of a signal generator of a medical device; emitting the generated monitoring signal, wherein the monitoring signal is emitted via an electrode, connected to the signal generator, of an invasive component of the medical device, wherein the electrode is arranged at a distance to a nerve, and wherein the monitoring signal triggers a motor and/or sensory reaction with action on the nerve, the form of which reaction depends on the distance of the electrode; detecting the form of the motor and/or sensory reaction, wherein the form of the motor and/or sensory reaction is detected by means of a detection apparatus of the medical device and/or by medical personnel; monitoring the distance of the electrode depending on the detected form of the motor and/or sensory reaction. The advantages accompanying the method according to the present disclosure correspond to the advantages of the medical device according to the present disclosure. To avoid repetitions, reference is made to the disclosure of the medical device in this regard, which also applies mutatis mutandis to the method according to the present disclosure. Further embodiments of the method according to the present disclosure result from the features of the medical device according to the present disclosure and its embodiments.

### BRIEF DESCRIPTION OF THE DRAWINGS

Further advantages and features of the present disclosure result from the following description of preferred exemplary embodiments of the present disclosure, which are illustrated on the basis of the drawings.

FIG. 1 shows a schematic block diagram of an embodiment of a medical device according to the present disclosure which is intended for use in pain therapy;

FIG. 2 shows a schematically simplified diagram to illustrate properties of electrical signals generated and emitted by means of the medical device; and

FIG. 3 shows a schematic block diagram of an embodiment of a method according to the present disclosure for monitoring the distance of an electrode during pain therapy.

### DETAILED DESCRIPTION

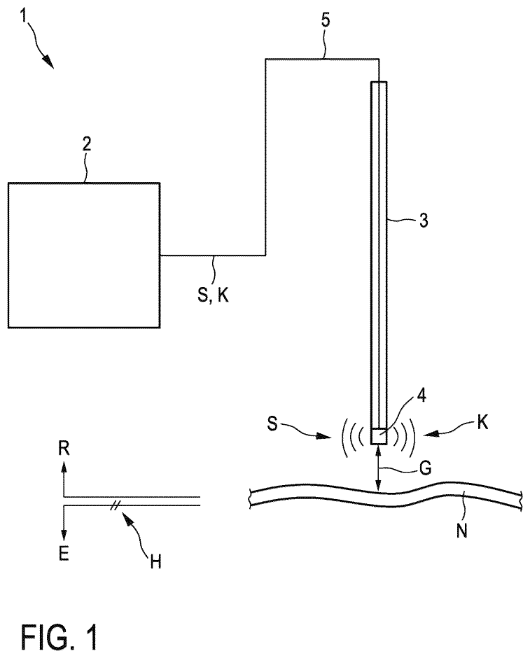

According to FIG. 1, a medical device 1 is intended for use in pain therapy and comprises a signal generator 2, an invasive component 3 and an electrode 4.

The signal generator 2 is configured to generate an electrical stimulation signal S and an electrical monitoring signal K. The electrode 4 is configured to emit the stimulation signal S and the monitoring signal K. The signal generator 2 and the electrode 4 are connected to one another by means of a signal line 5. The signal line 5 is used to transmit the generated signals S, K from the signal generator 2 to the electrode 4. The signal line 5 can be a wired or a wireless transmission line, wherein the wired transmission is preferred.

The electrode 4 is arranged and/or formed at a distal end (not designated in more detail) of the invasive component 3. The invasive component 3 can so to speak be understood as the electrode 4 and vice versa. In other words: The invasive component 3 is optional and is not present in all embodiments. In the simplest case, the medical device only comprises the signal generator and the electrode, which can be designed, for example, in the form of a signal wire, which is elongated starting from the signal generator.

The invasive component 3 including the electrode 4 is configured to be introduced into the body of a patient. The invasive component can be, for example, a catheter, a stimulation cannula or the like. In contrast, the signal generator 2 is an extracorporeal component of the medical device 1 and is therefore configured to remain outside the patient body.

In the use of the medical device 1, the electrode 4 is positioned in the area of a nerve N. In the exemplary usage situation shown in FIG. 1, the electrode 4 is positioned at a distance G from the nerve N.

The stimulation signal S generated by means of the signal generator 2 and emitted by means of the electrode 4 is configured to inhibit a stimulation transmission E of the nerve N. Said inhibition H is shown schematically in very simplified form in FIG. 1 as a type of interruption of the stimulation transmission E. The inhibition H of the stimulation transmission E causes an alleviation or else suppression of pain.

The monitoring signal K is configured to trigger a motor reaction R with action on the nerve N. A form or else strength of the motor reaction depends on the distance G between the electrode 4 and the nerve N. This applies accordingly to the effectiveness of the alleviation of pain by means of the stimulation signal S. An excessively strong reduction of the distance G can have the result that electrical energy transmitted by means of the stimulation signal S to the nerve N becomes too large. Vice versa, an excessively strong increase of the distance G can have the result that the electrical stimulation energy of the stimulation signal S becomes too low. Both can result in impairment of the effectiveness of the pain therapy.

The medical device 1 permits monitoring of the distance G, in particular in that the motor reaction R is detected and the distance G is adjusted accordingly. The detection of the motor reaction R can be carried out in the simplest case by medical personnel, i.e. a user of the medical device 1. On the basis of the observed motor reaction R, more precisely: its form/strength, the user can judge whether the electrode 4 has to be moved relative to the nerve N to achieve an optimum therapy effect, in order to optimize the distance G and the inhibition H, which is dependent on the distance G. Alternatively or additionally, the therapy effect can be adapted by an adaptation, in particular amplification, of the stimulation signal S.

In one embodiment, the medical device comprises a detection apparatus, which is configured for sensor-based detection of the motor reaction, especially its form, and for generation of a signal, which represents the form of the detected motor reaction. The distance can be adjusted accordingly depending on the signal generated by means of the detection apparatus.

However, there are also applications in which solely sensory nerves are stimulated. Monitoring by a motor reaction cannot take place in the case of neuromodulation of solely sensory nerves. In this case, reference can be made instead to a sensory reaction and accordingly to a “sensory monitoring signal”. The position monitoring does not take place in this case on the basis of the motor reactions, but rather on the basis of the patient feedback with regard to a sensory perception caused by the monitoring signal. The patient perceives the (sensory) monitoring signal and gives corresponding feedback to the attending anaesthetist: if the patient perceives nothing, the distance/the intensity is incorrectly selected. If the patient perceives the monitoring signal excessively strongly, the intensity is reduced/the distance is increased. The terms “sensory monitoring signal” and “motor monitoring signal” are summarized in the scope of this disclosure under the term “monitoring signal”, unless described otherwise.

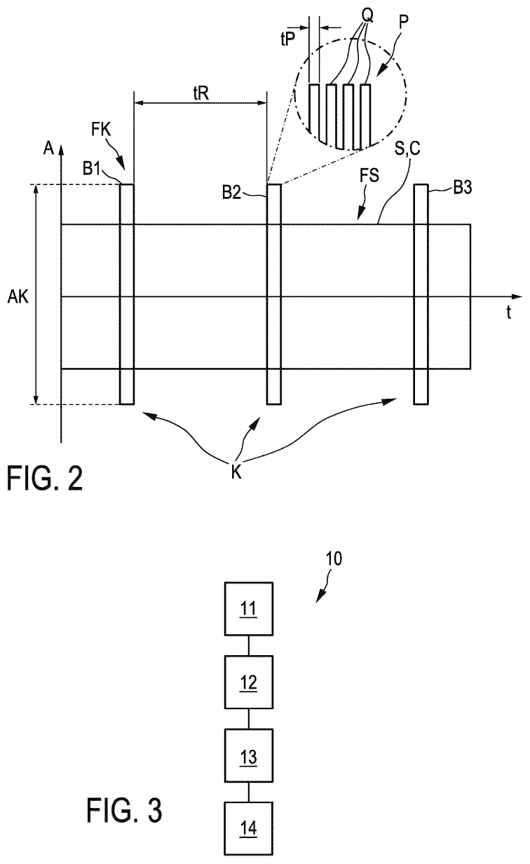

The stimulation signal S and the monitoring signal K have different properties, in particular different electrical and/or different signalling properties. These different properties will be explained hereinafter with reference to FIG. 2.

FIG. 2 shows a schematically very simplified time course of the two signals S, K, wherein, in the diagram shown, an amplitude A is shown over time t. In the present case, the amplitude A refers to an amperage of the respective signals S, K.

In the embodiment shown, the monitoring signal K is a low-frequency signal having a frequency FK. The stimulation signal S is a high-frequency signal having a frequency FS. The frequency FS is approximately 2.5×10³ greater than the frequency FK of the monitoring signal K in the embodiment shown. In the present case, the frequency FK of the monitoring signal K is especially 1 Hz. The frequency FS of the stimulation signal is 25 kHz in the present case.

In one embodiment, the stimulation signal has a frequency of 0.1 Hz to 200 Hz, preferably of 0.5 Hz to 50 Hz, more preferably of 3 Hz to 5 Hz, in particular wherein the stimulation signal has a signal strength which lies below the perception threshold.

Furthermore, an amplitude AK of the monitoring signal K is 1.5 mA in the present case. In some embodiments, the amplitude of the monitoring signal is between 0.01 mA and 20 mA.

As is furthermore shown in FIG. 2, the stimulation signal S is a continuous signal C. In contrast, the monitoring signal K is a pulsed signal P. In other words: The stimulation signal S is emitted continuously or in any case quasi-continuously over time t. In contrast, the monitoring signal K is generated and emitted in a pulsed or else cycled manner and/or in a manner provided with chronological interruptions.

As is shown in the enlarged area in FIG. 2, a pulse width tP of the monitoring signal K is 0.5 ms in the present case. In some embodiments, the pulse width of the monitoring signal is from 0.01 ms to 20 ms.

As is furthermore shown in FIG. 2, the monitoring signal K is generated and emitted in multiple bursts or in other words intermittently, wherein in the present case multiple bursts B1, B2, B3 are shown by way of example. These can also be designated as first burst B1, second burst B2 and third burst B3. It is apparent that the number of the bursts shown is solely by way of example. Depending on the duration of the pain therapy, of course, more or even significantly more than the three bursts shown by way of example in the present case can also be generated and emitted.

Each of the bursts B1, B2, B3 shown comprises a defined number of pulses Q, wherein in the present case four pulses are emitted per burst. This number of pulses is also exemplary. In some embodiments, the number of pulses per burst is one to one hundred pulses, preferably one to ten pulses, more preferably one to five pulses.

A pause duration tR is provided between each of the bursts B1, B2, B3. This is 20 s in the present case. In some embodiments, the pause duration is at least 10 s, preferably at least 15 s and more preferably at least 20 s.

FIG. 3 shows in a schematically simplified manner a method 10 for monitoring the distance of the electrode 4 during the use of the medical device 1. The method provides generating 11 the electrical monitoring signal K, wherein the monitoring signal K is generated by means of the signal generator 2. The method furthermore provides emitting 12 the generated monitoring signal K, wherein the monitoring signal K is emitted via the electrode 4 of the invasive component 3, which is connected by means of the signal line 5 to the signal generator 2. The electrode 4 is arranged here at the already mentioned distance G to the nerve N. The monitoring signal K triggers, with action on the nerve N, the already mentioned motor and/or sensory reaction R, the form or else strength of which depends on the distance G. The method furthermore provides detecting 13 the form of the motor and/or sensory reaction R. The detection 13 is carried out either by means of the abovementioned optional detection apparatus or by the medical personnel themselves. Furthermore, the method 10 provides monitoring 14 the distance G depending on the detected form of the motor and/or sensory reaction R. The monitoring 14 is carried out in the simplest case by the medical personnel themselves. Alternatively, the medical device can comprise an optional monitoring apparatus which automatically monitors and optionally adjust the distance. This monitoring and optional adjustment can take place, for example, depending on a signal which is generated by means of the optional detection apparatus and represents the form of the motor and/or sensory reaction.

The following Table shows value ranges that have been found to be advantageous for the pulse width, the frequency and the amperage of the monitoring signal and the stimulation signal in further embodiments:

TABLE

| | | monitoring signal | | | | |
|---|---|---|---|---|---|---|
| | | sensory | motor | stimulation signal | | |
| | | reaction | reaction | high-frequency | | low-frequency |
| pulse width | | 0.01 ms-20 ms | 0.01 ms-20 ms | — | | 0.01 ms-250 ms |
| | preferably | 0.05 ms-10 ms | 0.05 ms-10 ms | | | 0.1 ms-100 ms |
| | more preferably | 0.1 ms-0.5 ms, | 0.1 ms-0.5 ms | | | 0.1 ms-0.5 ms, |
| | | ideally 0.2 ms | | | | ideally 0.2 ms |
| frequency | | 0.1 Hz-100 Hz | 0.1 Hz-10 Hz | >5 | kHz | 0.1 Hz-200 Hz |
| | preferably | 0.5 Hz-50 Hz | 0.5 Hz-5 Hz | >10 | kHz | 0.5 Hz-50 Hz |
| | more preferably | 3-5 Hz | 1-2 Hz | >20 | kHz | 3-5 Hz |
| amperage | | 0.03 mA-60 mA | 0.01 mA-20 mA | — | | less than the |
| | preferably | 0.3 mA-30 mA | 0.1 mA-19 mA | | | monitoring |
| | more preferably | 1.5 mA-9 mA | 0.5 mA-3 mA | | | signal |

1. A medical device for use in pain therapy, the medical device comprising:
an invasive component having at least one electrode configured for positioning at a nerve and for emitting an electrical stimulation signal; and
a signal generator connected to the at least one electrode and configured to generate the electrical stimulation signal,
the electrical stimulation signal configured to inhibit a stimulation transmission of the nerve,
the at least one electrode configured to emit an electrical monitoring signal,
the signal generator configured to generate the electrical monitoring signal,
the electrical monitoring signal configured to trigger a motor reaction and/or a sensory reaction, and
the motor reaction and/or the sensory reaction depending on a distance between the at least one electrode and the nerve.

2. The medical device according to claim 1, wherein:
the electrical stimulation signal is not configured to trigger the motor reaction and/or the sensory reaction, and
the electrical monitoring signal is not configured to inhibit the stimulation transmission of the nerve.

3. The medical device according to claim 1, wherein a frequency of the electrical stimulation signal is greater than a frequency of the electrical monitoring signal by a factor of at least 0.5×10³.

4. The medical device according to claim 1, wherein a frequency of the electrical stimulation signal is greater than a frequency of the electrical monitoring signal by a factor of 1.0×10³.

5. The medical device according to claim 1, wherein a frequency of the electrical stimulation signal is greater than a frequency of the electrical monitoring signal by a factor of 1.5×10³.

6. The medical device according to claim 1, wherein the electrical monitoring signal has a frequency of 0.1 Hz to 10 Hz.

7. The medical device according to claim 1, wherein the electrical monitoring signal has a frequency of 0.5 Hz to 5 Hz.

8. The medical device according to claim 1, wherein the electrical monitoring signal has a frequency of 1 Hz to 2 Hz.

9. The medical device according to claim 1, wherein the electrical stimulation signal has a frequency of at least 5 kHz.

10. The medical device according to claim 1, wherein the electrical stimulation signal has a frequency of 0.1 Hz to 200 Hz.

11. The medical device according to claim 1, wherein the electrical monitoring signal has an amplitude of 0.01 mA to 20 mA.

12. The medical device according to claim 1, wherein the signal generator is configured to generate the electrical monitoring signal as a pulsed signal and to generate the electrical stimulation signal as a pulsed signal.

13. The medical device according to claim 1, wherein the signal generator is configured to generate the electrical monitoring signal as a pulsed signal and to generate the electrical stimulation signal as a continuous signal.

14. The medical device according to claim 13, wherein the electrical monitoring signal has a pulse width of 0.01 ms to 20 ms.

15. The medical device according to claim 13, wherein the signal generator is configured to generate the electrical monitoring signal in at least one burst having a number of pulses of 1 to 100 pulses.

16. The medical device according to claim 13, wherein the signal generator is configured to generate the electrical monitoring signal in at least one burst having a number of pulses of 1 to 10 pulses.

17. The medical device according to claim 16, wherein the signal generator is configured to generate the electrical monitoring signal in at least two successive bursts, wherein a pause duration between the at least two successive bursts is at least 10 s.

18. The medical device according to claim 1, wherein the signal generator is configured to generate the electrical monitoring signal and the electrical stimulation signal alternately.

19. The medical device according to claim 1, wherein the signal generator is configured to generate the electrical monitoring signal and the electrical stimulation signal simultaneously.

20. A method for distance monitoring of an electrode during pain therapy by electrical neurostimulation, the method comprising the steps of:
generating an electrical monitoring signal with a signal generator of a medical device;
emitting the electrical monitoring signal via an electrode, the electrode connected to the signal generator of an invasive component of the medical device, the electrode arranged at a distance from a nerve, and the electrical monitoring signal triggering a motor reaction and/or a sensory reaction with action on the nerve, a form of the motor reaction and/or the sensory reaction depending on the distance from the nerve;
detecting the form of the motor reaction and/or the sensory reaction by a detection apparatus of the medical device and/or by medical personnel;
monitoring the distance from the nerve depending on the form of the motor reaction and/or the sensory reaction.
